(19) United States
(12) Patent Application Publication
(10) Pub. No.: US 2023/0191306 A1
(43) Pub. Date: Jun. 22, 2023

(54) BREATHING TUBE FILTER AND FILTER ASSEMBLY

(71) Applicant: SDI Diagnostics, Inc., Easton, MA (US)

(72) Inventor: Michael BOYLE, Easton, MA (US)

(21) Appl. No.: 18/069,844

(22) Filed: Dec. 21, 2022

Related U.S. Application Data
(60) Provisional application No. 63/293,001, filed on Dec. 22, 2021.

(51) Int. Cl. B01D 46/52; B01D 46/00; B01D 46/10; A61B 5/087

(52) U.S. Cl. CPC B01D 46/521; B01D 46/0032; B01D 46/10; B01D 46/0012; A61B 5/087; B01D 2271/027

(57) ABSTRACT

A pulmonary function test filter and filter assembly for use with a breathing tube of a spirometer. The filter assembly includes a first portion having an exhaust end configured to removably engage an exhaust end of a flow tube of a spirometer, preferably without an adapter, a second portion configured to cooperatively engage the first portion and including a mouthpiece, and filter media. The filter media is removably securable between the first and second portions.

### CROSS REFERENCE TO RELATED APPLICATION

This application claims the benefit of priority to U.S. Provisional Patent Application No. 63/293,001, filed Dec. 22, 2021, the disclosure of which is incorporated by reference herein in its entirety.

### FIELD OF THE INVENTION

The invention relates to breathing tube filters, and more particularly, breathing tube filters for use with ultrasonic spirometers.

### BACKGROUND OF THE INVENTION

A spirometer is an apparatus for measuring the volume of air inspired and expired by the lungs of a patient. A spirometer measures ventilation, i.e., the movement of air into and out of the lungs. The spirogram identifies two different types of abnormal ventilation patterns: obstructive and restrictive. There are various types of spirometers that use a number of different methods for measurement (e.g., pressure transducers, ultrasonic, water gauge).

A spirometer is the main piece of equipment used for basic Pulmonary Function Tests (PFTs). Lung diseases such as asthma, bronchitis, and emphysema may be ruled out from the results of the PFTs. In addition, a spirometer often is used for finding the cause of shortness of breath, assessing the effect of contaminants on lung function, the effect of medication, and evaluating progress for disease treatment.

In operation, a medical professional instructs a test subject/patient to exhale forcefully into the breathing tube. The expired gases are directed across the ultrasonic transmitter/receiver system in the spirometer. This system is designed to measure the flow by transit times with the flow and against the flow. By forcefully blowing into the breathing tube there exists a possibility of expelling harmful pathogens into the test area environment. Prior art has no mechanism to prevent this potentially very dangerous occurrence.

Spirometers use disposable mouthpieces to eliminate cross-contamination of test subjects. Many of the mouthpieces contain viral/bacterial filters that offer an extra layer of protection for the patient and the test administrator. Spirometry involves exhaling forcefully into the device. Unless a filter is used, any viruses or bacteria from the patient are blown into the test environment. This can be very unsafe for the medical professional(s) treating the patient and any others in the surrounding area.

One model of spirometer is made in Switzerland by Ndd Medizintechnik, AG. This spirometer uses a “breathing tube” as the mouthpiece and is disclosed in U.S. Pat. No. 10,786,178, the disclosure of which is incorporated by reference herein in its entirety. The shape of the breathing tube disclosed in the ′178 Patent is intended to only allow correct insertion of the breathing tube into the spirometer. This is accomplished by utilizing a “polygonal transversal cross section along the longitudinal axis of the breathing tube” which can only be used in the correct orientation. The breathing tube of the ′178 Patent also has at least a pair of beveled outer sides opposing on another and extending longitudinal axis.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In one aspect of the innovation, a filter assembly comprises a first portion including an exhaust end configured to removably engage an exhaust end of a flow tube of a spirometer, a middle section configured to removably engage a mouthpiece of the spirometer flow tube, and a sealing ring; and a second portion configured to cooperatively engage the first portion and including a mouthpiece.

In another aspect of the innovation, a filter assembly comprises a first portion including an exhaust end configured to removably engage an exhaust end of a flow tube of a spirometer; a second portion configured to cooperatively engage the first portion and including a mouthpiece; and filter media, wherein the filter media is removably securable between the first and second portions.

In yet another aspect of the innovation, a filter assembly comprises a first portion including an exhaust end configured to removably engage an exhaust end of a flow tube of a spirometer, a middle section configured to removably engage a mouthpiece of the spirometer flow tube, a sealing ring and a first filter surface opposite the exhaust end; a second portion configured to cooperatively engage the first portion and including a mouthpiece and a complimentary-shaped second filter surface opposite the mouthpiece; and filter media, wherein the filter media is removably securable between the first filter surface and the second filter surface.

### BRIEF DESCRIPTION OF THE DRAWINGS

Embodiments of the invention are further described but are in no way limited by the following illustrations.

FIG. 1 is a top perspective view of a breathing tube filter according to the present invention, as used with a breathing tube; and

FIG. 2 is an exploded top perspective view of the breathing tube filter and filter tube of FIG. 1.

### DETAILED DESCRIPTION OF THE INVENTION

For clarity and convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

As used in this specification and the appended claims, the singular forms “a,” “an” and “the” include plural referents unless the content clearly dictates otherwise.

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

Disclosed herein is a pulmonary function test filter and filter assembly for use with a breathing tube of a spirometer.

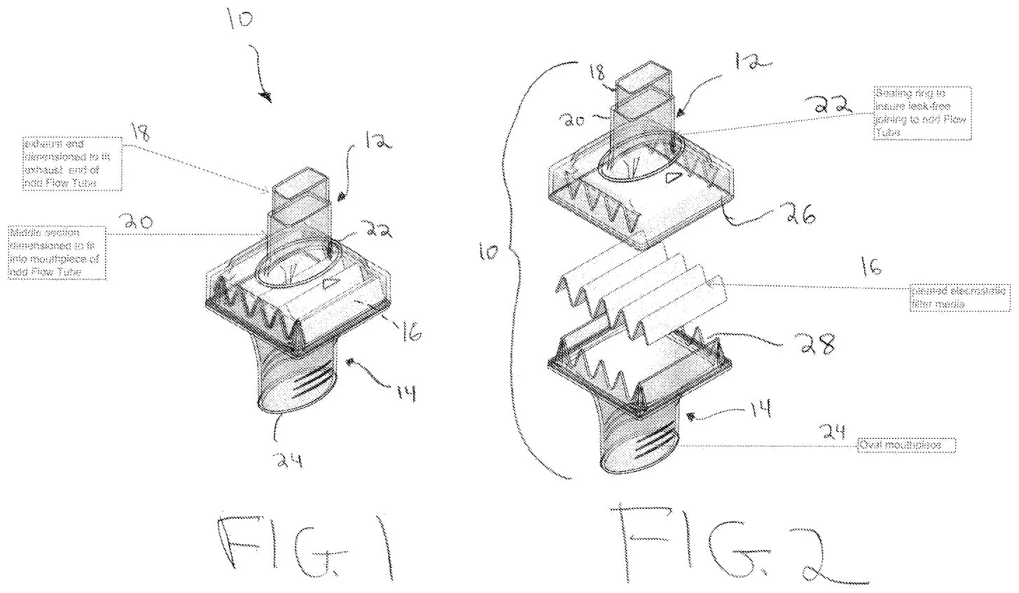

An embodiment of the pulmonary function filter and filter assembly of the present invention is illustrated in FIGS. 1 and 2. A pulmonary function filter assembly 10 includes a first portion 12, a second portion 14 configured to cooperatively engage the first portion 12, and a pleated electrostatic filter media 16 that is removably positionable between the first and second portions 12, 14.

The first portion 12 includes an exhaust end 18 that is configured (e.g., dimensioned) to fit on an exhaust end of a flow tube of a spirometer (e.g., an NDD spirometer flow tube (not shown)), a middle section 20 that is configured (e.g., dimensioned) to fit into the mouthpiece of the NDD spirometer flow tube, and a sealing ring 22 to ensure a leak-free juncture with the NDD spirometer flow tube. The second portion 14 includes an oval mouthpiece 24.

The pleated electrostatic filter media 16 is securable between the first and second portions 12, 14. As shown in FIGS. 1 and 2, the first and second filter surfaces 26, 28, and electrostatic filter media 16, all have a zig-zag shape. Other shapes are possible in other embodiments.

As explained above and illustrated in FIGS. 1 and 2, the filter assembly 10 is configured to be used on the mouthpiece/input end of the spirometer or on the exhaust end of the spirometer without an adapter. Thus, the medical professional using the spirometer to administer a PFT has the choice of how to use the filter assembly with the spirometer, based on their best judgement and other applicable factors.

In the embodiment illustrated in FIGS. 1 and 2, the filter assembly 10 is configured for use with a pleated electrostatic filter media 16. In alternate embodiments, the filter assembly 10 is configured for sue with other types of filters/filter media. Such filters include those having a disk form/format.

In various embodiments, the filter assembly 10 can be formed from various suitable materials. Such materials include, but are limited to, injection molded polypropylene.

In general, any combination of disclosed features, components and methods described herein is possible. Steps of a method can be performed in any order that is physically possible.

Although embodiments have been disclosed, the invention is not limited thereby.

I claim:

1. A filter assembly comprising:
a first portion including an exhaust end configured to removably engage an exhaust end of a flow tube of a spirometer, a middle section configured to removably engage a mouthpiece of the spirometer flow tube, and a sealing ring; and
a second portion configured to cooperatively engage the first portion and including a mouthpiece.

2. The filter assembly of claim 1, further comprising filter media, wherein the filter media is removably securable between the first and second portions.

3. The filter assembly of claim 2, wherein the first portion further includes a first filter surface opposite the exhaust end, and the second portion further includes a complimentary-shaped second filter surface opposite the mouthpiece, whereby the filter media is removably securable between the first filter surface and the second filter surface.

4. The filter assembly of claim 3, wherein the first and second filter surfaces and filter media all have a zig-zag shape.

5. The filter assembly of claim 2, wherein the filter media is pleated electrostatic filter media.

6. The filter assembly of claim 2, wherein the filter media is disk form filter media.

7. The filter assembly of claim 1, wherein the mouthpiece has an oval cross section.

8. The filter assembly of claim 1, wherein the exhaust end of the first portion is configured to removably engage the exhaust end of the spirometer flow tube without an adapter.

9. The filter assembly of claim 1, wherein the first portion and the second portion are formed from injection molded polypropylene.

10. A filter assembly comprising:
a first portion including an exhaust end configured to removably engage an exhaust end of a flow tube of a spirometer;
a second portion configured to cooperatively engage the first portion and including a mouthpiece; and
filter media, wherein the filter media is removably securable between the first and second portions.

11. The filter assembly of claim 10, wherein the first portion further includes a first filter surface opposite the exhaust end, and the second portion further includes a complimentary-shaped second filter surface opposite the mouthpiece, whereby the filter media is removably securable between the first filter surface and the second filter surface.

12. The filter assembly of claim 11, wherein the first and second filter surfaces and filter media all have a zig-zag shape.

13. The filter assembly of claim 10, wherein the first portion further includes a middle section configured to removably engage a mouthpiece of the spirometer flow tube.

14. The filter assembly of claim 10, wherein the first portion further includes a sealing ring.

15. The filter assembly of claim 10, wherein the mouthpiece has an oval cross section.

16. The filter assembly of claim 10, wherein the exhaust end of the first portion is configured to removably engage the exhaust end of the spirometer flow tube without an adapter.

17. The filter assembly of claim 10, wherein the first portion and the second portion are formed from injection molded polypropylene.

18. The filter assembly of claim 10, wherein the filter media is pleated electrostatic filter media.

19. The filter assembly of claim 10, wherein the filter media is disk form filter media.

20. A filter assembly comprising:
a first portion including an exhaust end configured to removably engage an exhaust end of a flow tube of a spirometer, a middle section configured to removably engage a mouthpiece of the spirometer flow tube, a sealing ring and a first filter surface opposite the exhaust end;
a second portion configured to cooperatively engage the first portion and including a mouthpiece and a complimentary-shaped second filter surface opposite the mouthpiece; and
filter media, wherein the filter media is removably securable between the first filter surface and the second filter surface.
